(19) United States
(12) Patent Application Publication
(10) Pub. No.: US 2009/0111120 A1
(43) Pub. Date: Apr. 30, 2009

(54) METHYLATION OF GENES AS A PREDICTOR OF POLYP FORMATION AND RECURRENCE

(75) Inventors: Stephen J. Meltzer, Lutherville, MD (US); Zhe Jin, Towson, MD (US); Fumiaki Sato, Ukyo-ku (JP); Bogdan Constantin Paun, Baltimore, MD (US)

Correspondence Address: SUGHRUE MION, PLLC, 2100 PENNSYLVANIA AVENUE, N.W., SUITE 800, WASHINGTON, DC, 20037, US

(73) Assignee: UNIVERSITY OF MARYLAND, BALTIMORE, Baltimore, MD (US)

(21) Appl. No.: 12/295,626

(22) PCT Filed: Mar. 30, 2007
(86) PCT No.: PCT/US07/65696

§ 371 Date: Nov. 25, 2008

Related U.S. Application Data
(60) Provisional application No. 60/743,999, filed on Mar. 30, 2006.

(51) Int. Cl. C12Q 1/68

(52) U.S. Cl. 435/6

(57) ABSTRACT

The present invention provides methods for identifying or assessing probabilities for developing an abnormal condition in subject and for the recurrence of the abnormal condition in the subject after receiving treatment. The method comprises determining the methylation status of at least one gene in the subject and comparing this methylation status to normal methylation status. Differences between the methylation status of the one or more genes is indicative of the subject developing an abnormal condition or for the recurrence of the abnormal conditions after receiving treatment.

### CROSS-REFERENCE TO RELATED APPLICATIONS

This application claims priority to U.S. Provisional Application No. 60/743,999, filed 30 Mar. 2006, which is incorporated by reference.

### STATEMENT REGARD FEDERALLY SPONSORED RESEARCH OR DEVELOPMENT

Part of the work performed during development of this invention utilized U.S. Government funds under NIH Grants CA77057 and CA95323. The U.S. Government has certain rights in this invention.

### FIELD OF THE INVENTION

The present invention provides methods for identifying or assessing probabilities for developing an abnormal condition in subject and for the recurrence of the abnormal condition in the subject after receiving treatment. The method comprises determining the methylation status and level of at least one gene in the subject and comparing this methylation status and level to normal methylation status and level. Differences between the methylation status or level of these one or more genes is indicative of a high risk of the subject having or developing an abnormal condition or of recurrence of the abnormal condition after receiving treatment.

### BACKGROUND OF THE INVENTION

Abnormal methylation of DNA (hypermethylation or hypomethylation) plays a role in gene activity, cell differentiation, tumorigenesis, X-chromosome inactivation, genomic imprinting and other major biological processes (See Razin, A., H., and Riggs, R. D. eds. in DNA Methylation Biochemistry and Biological Significance, Springer-Verlag, N.Y., 1984). In eukaryotic cells in general, methylation of cytosine residues that are immediately 5′ to a guanosine, occurs predominantly in cytosine-guanine (CG)-poor regions (See Bird, Nature, 321:209, 1986). In contrast, CG-rich regions (so-called “CpG islands”) are generally unmethylated in normal cells, except during X-chromosome inactivation and parental-specific imprinting (Li, et al., Nature, 366:362, 1993), where methylation of 5′ regulatory regions can lead to transcriptional repression. For example, a detailed analysis of the VHL gene showed aberrant methylation in a subset of sporadic renal cell carcinomas (Herman, et al., Proc. Natl. Acad. Sci., U.S.A., 91:9700, 1994).

The precise role of abnormal DNA methylation, however, in human tumorigenesis has not been fully established. About half of the tumor suppressor genes which have been shown to be mutated in the germline of patients with familial cancer syndromes have also been shown to be aberrantly methylated in some proportion of sporadic cancers, including APC, Rb, VHL, p16,hMLH1, and BRCA1 (reviewed in Baylin, et al., Adv. Cancer Res. 72:141-196 1998). Methylation of tumor suppressor genes in cancer is usually associated with (1) lack of gene transcription and (2) absence of coding region mutation. Thus CpG island methylation can serve as an alternative mechanism of gene inactivation (silencing) in human cancers.

Expression of a tumor suppressor gene can be diminished or ablated by de novo DNA methylation of a normally unmethylated CpG island (Issa, et al., Nature Genet., 7:536, 1994; Merlo, et al., Nature Med., 1:686, 1995 and Herman, et al., Cancer Res., 56:722, 1996). Methylation of tumor-suppressor genes leads to the reduced expression of tumor suppressor genes, resulting in unchecked cellular growth, tissue invasion, angiogenesis, and metastases (See Das, P. M. and Singal, R. J Clin Oncol, 22: 4632-4642 (2004) and Momparler, R. L. Oncogene, 22: 6479-6483 (2003)). Indeed, multiple studies have shown that promoter hypermethylation of tumor suppressor genes may also underlie carcinogenesis (See Eads, C. A., et al., Cancer Res., 61:3410-3418 (2001), Sato, F. et al. Cancer Res., 62: 6820-6822 (2002) and Takahashi, T., et al, Int J Cancer, 115:503-510 (2005), all of which are incorporated by reference). In addition, aberrant methylation across panels of genes correlates with prognosis in many cancers (See Darnton, S. J., et al., Int J Cancer, 115:351-358(2005), Kawakami, K., et al., J Natl Cancer Inst, 92:1805-1811 (2000), Kikuchi, S., et al., Clin Cancer Res, 11:2954-2961 (2005) and Catto, J. W., et al., J Clin Oncol, 23:2903-2910 (2005), all of which are incorporated by reference). Indeed, prior studies have validated analyzing methylation patterns across a panel of genes to predict prognosis in esophageal and rectal cancers (See Brock, M. V., et al, Clin Cancer Res, 9:2912-2919 (2003), Ghadimi, B. M., et al, J Clin Oncol, 23:1826-1838 (2005), both incorporated by reference). Furthermore, human cancer cells typically contain nucleic acids that display somatic changes in DNA methylation (Makos, et al, Proc. Natl. Acad. Sci., USA, 89:1929, 1992; Ohtani-Fujita, et al., Oncogene, 8:1063, 1993).

Conversely, diminished DNA methylation (hypomethylation) has also been described in numerous human malignant and premalignant conditions (Martinez M E et al., Gastroenterology 2006 Dec;131(6):1706-16; Cadieux B et al., Cancer Res. 2006 Sep 1;66(17):8469-76; Rodriquez J et al., Cancer Res. 2006 Sep 1;66(17):8462-8; Ehrlich M, Curr Top Microbiol Immunol 2006:310:251-74). This abnormally low level of methylation may lead to the activation, or abnormally high expression, of tumor-promoting genes or microRNAs, such as oncogenes and oncomiRs (Brueckner et al., Cancer Res. 2007 Feb 15:67(4):1419-23; Lujambio A et al., Cancer Res. 2007 Feb. 15;67(4):1424-9. Thus, there is a role for hypomethylation in the genesis and/or progression of human cancers.

Despite the abundance of evidence that characterizes certain molecular events in colorectal cancer initiation, promotion and progression, the incidence of colorectal cancer in the United States is rising. New tests and diagnostics are needed to better evaluate which patients are most at risk for developing colorectal polyps and cancers, or for the likelihood of their recurrence after initial treatment.

### SUMMARY OF THE INVENTION

The present invention provides methods for identifying or assessing probabilities for the recurrence of an abnormal condition in a subject. The method comprises determining the methylation status and level of at least one gene in the subject and comparing this methylation status or level to normal methylation status. Differences between the methylation status or level of these one or more genes is indicative of the recurrence of the abnormal condition, such as colon polyps in the subject.

The present invention also provides methods for identifying or assessing probabilities of developing an abnormal condition in a subject. The method comprises determining the methylation status and level of at least one gene in the subject and comparing this methylation status or level to normal methylation status or level. Differences between the methylation status or level of these one or more genes is indicative of the probability of developing an abnormal condition, such as colon polyps in the subject.

The present invention also provides methods of individualizing a therapeutic regimen for a subject in need thereof, with the methods comprising determining the methylation status or level of a gene or panel of genes in a test subject and using the methylation status or level in the test subject to dictate a therapeutic regimen. Based upon said test subject's methylation status, a health care provider can then determine an appropriate therapeutic regimen going forward.

The present invention also provides methods for assessing the probability of a subject having an abnormal condition, with the methods comprising determining a methylation status of at least one gene in gross normal tissue of the subject and comparing the methylation status of the gene or genes in said subject to the normal methylation status of the at least one gene. Differences between the methylation status of the at least one gene in the gross normal tissue of the subject and the normal methylation status of the at least one gene indicates that the subject has an altered probability of having said abnormal condition.

### BRIEF DESCRIPTION OF THE DRAWINGS

FIG. 1 depicts the ROC curve based on dataset composed of APC, MGMT, MLH1, NELL 1, and RARβ. This dataset exhibited the best AUROC using linear discriminant analysis and leave-one-out crossvalidation vs. polyp recurrence. Methylation of MLH1, NELL1, and RARβ correlated inversely with adenoma recurrence. A cutoff value of 5% methylation was set prior statistical analysis to define positive vs. negative methylation in the index sample. ROC curve analyses were performed using Analyse-It+Clinical Laboratory 1.71. AUROC=0.7434.

FIG. 2 depicts the ROC curve based on dataset composed of age, APC, MLH1, pl6, RARβ, and biggest polyp size. This dataset exhibited the best AUROC using linear discriminant analysis and leave-one-out crossvalidation vs. the presence of a concurrent adenoma at the same time as the index polypectomy. Methylation of MLH1, RARβ and biggest polyp size correlated inversely with adenoma concurrence. A cutoff value of 5% methylation was set prior to statistical analysis define positive vs. negative methylation in the index sample. ROC curve analyses were performed using Analyse-It+Clinical Laboratory 1.71. AUROC=0.6929.

FIG. 3 depicts ROC curve based on dataset composed of age, APC, NELL1, p14, and methylation index (composed of APC, ESR1, HPP1, MGMT, p14, p15, RAR_{.}⁻, and TAC1). This dataset exhibited the best AUROC using linear discriminant analysis and leave-one-out crossvalidation vs. the presence of a concurrent adenoma at the time of index polypectomy. A cutoff value of 5% methylation was set a priori to define positive vs. negative methylation in the index sample. ROC curve analyses were performed using Analyse-It+Clinical Laboratory 1.71. AUROC=0.6661.

### DETAILED DESCRIPTION OF THE INVENTION

The present invention provides methods for identifying or assessing probabilities for the presence, recurrence or development of an abnormal condition in subject. As used herein, “predicting” or “assessing the probability” indicates that the methods described herein are designed to provide information to a health care provider or computer, to enable the health care provider or computer to determine the likelihood that an abnormal condition is already present, may occur in the future, or may recur in the future in a subject. Examples of health care providers include but are not limited to, an attending physician, oncologist, physician's assistant, pathologists, laboratory technician, etc. The information may also be provided to a computer, where the computer comprises a memory unit and machine-executable instructions that are configured to execute at least one algorithm designed to determine the likelihood that an abnormal condition may be already present, may occur in the future, or may recur in the future in a subject. Accordingly, the invention also provides devices for predicting the likelihood of current presence, future occurrence, or future recurrence of an abnormal condition in a subject, comprising a computer with machine-executable instructions for predicting the likelihood of presence, occurrence, or recurrence.

As used herein, the term “subject” is used interchangeably with the term “patient,” and is used to mean an animal, in particular a mammal, and even more particularly a non-human or human primate.

As used herein, a “recurrence” indicates that the abnormal condition occurs again in a patient, after the condition has been treated such that the condition is no longer detectable in the subject. The recurrence time for the abnormal condition resurfacing is not limited in any way. Furthermore, the term “treat” or “treatment” is used to indicate a procedure which is designed to ameliorate one or more causes, symptoms, or untoward effects of an abnormal condition in a subject. The treatment can, but need not, cure the subject, i. e., remove the cause(s), or remove entirely the symptom(s) and/or untoward effect(s) of the abnormal condition in the subject. The methods of the present invention can be performed prior to, in conjunction with, or after the treating the subject. Thus, for example, the methods of the present invention may be performed prior to treating the subject such that a more or less aggressive treatment strategy can be employed in the subject, if necessary. Accordingly, the present invention provides methods of individualizing treatments or therapeutic regimens in a subject by utilizing the methylation status or level of a gene or panel of genes. The phrase “therapeutic regimen” is used to indicate a procedure which is designed to terminate abnormal growth(s), inhibit growth and accelerate cell aging, induce apoptosis and cell death of neoplastic tissue within a subject. Additionally, “therapeutic regimen” means to reduce, stall, or inhibit the growth of or proliferation of tumor cells, including but not limited to precancerous or carcinoma cells. The therapeutic regimen may or may not be employed prior to performing the methods of the present invention. The invention is not limited by the therapeutic regimen contemplated. Examples of therapeutic regimens include but are not limited to chemotherapy (pharmaceuticals), radiation therapy, surgical intervention, endoscopic or colonoscopic excision, cell therapy, stem cell therapy, gene therapy and any combination thereof In one embodiment, the therapeutic regimen comprises chemotherapy. In another embodiment, the therapeutic regimen comprises radiation therapy. In yet another embodiment, the therapeutic regimen comprises surgical intervention. In still another embodiment, the therapeutic regimen comprises a combination of chemotherapy and radiation therapy. In still another embodiment, the therapeutic regimen comprises initial or repeat colonoscopy with or without polypectomy or removal of other abnormal growths.

Of course, the therapeutic regimen that is being employed or contemplated will depend on the abnormal condition that the subject has or is suspected of having. As used herein, an “abnormal condition” is used to mean a disease, or aberrant cellular or metabolic condition. Examples of abnormal conditions in which the methods can be used include but are not limited to, dysplasia, neoplastic growth and abnormal cell proliferation. In one embodiment, the abnormal condition comprises neoplastic growth. In a more specific embodiment, the abnormal condition comprises a colon polyp. The colon polyp may or may not be cancerous. The invention, however, is not necessarily limited to the type of neoplasm. For example, the neoplasm may be a carcinoma of the digestive tract or any associated glands or organs, including, but not limited to, the throat, the salivary glands, vocal cords, esophagus, the stomach, the small intestine, the large intestine, the pancreas, liver, gallbladder, biliary tree, and rectum. Additional forms of neoplasms include, but are limited to, cancer of the lung, prostate, ovary, urinary tract, and breast.

The methods comprise determining the methylation status and level of a gene or panel of genes in the test subject. As used herein, “methylation status” is used to indicate the presence or absence or the level or extent of methyl group modification in the polynucleotide of at least one gene. As used herein, “methylation level” is used to indicate the quantitative measurement of methylated DNA for a given gene, defined as the percentage of total DNA copies of that gene that are determined to be methylated, based on quantitative methylation-specific PCR. As used herein, a “panel of genes” is a collection of genes comprising 2 or more distinct genes. In one embodiment, the panel of genes comprises at least 2, 3, 4, 5, 6, 7, 8, 9, 10, 11, 12, 13, 14, 15, 16, 17, 18, 19 or 20 or more genes.

The term “gene” is used similarly to as it is in the art. Namely, a gene is a region of DNA that is responsible for the production and regulation of a polypeptide chain. Genes include both coding and non-coding portions, including introns, exons, promoters, initiators, enhancers, terminators, microRNAs, and other regulatory elements. As used herein, “gene” is intended to mean at least a portion of a gene. Thus, for example, “gene” may be considered a promoter for the purposes of the present invention. Accordingly, in one embodiment of the present invention, at least one member of the panel of genes comprises a non-coding portion of the entire gene. In a particular embodiment, the non-coding portion of the gene is a promoter. In another embodiment, all members of the entire panel of genes comprise non-coding portions of the genes, such as but not limited to, introns. In another particular embodiment, the non-coding portions of the members of the genes are promoters. In another embodiment of the present invention, at least one member of the panel of genes comprises a coding portion of the gene. In another embodiment, all members of the entire panel of genes comprise coding portions of the genes. In one particular embodiment, the coding portion of the gene is at the 5′ end of the coding portion of the gene. In another particular embodiment, the coding portion of the gene is at the 3′ end of the coding portion of the gene.

Candidate members of the gene panel include, but are not limited to, tumor suppressor genes, tumor promoter genes and other genes that may be involved in cell cycle regulation. Examples of genes involved in the regulation of cell cycle that could serve as members of the gene panel include, but are not limited to, Reprimo, p14, p15, p16, p27 CHFR, TIMP-3, MGMT, ESR1, NELL1, MLH1, APC, SST, TAC1, HPP1, HIN1, CDH1, GSTP1, RARβ, TAC1, and SST. The tumor genetics of genes have been evaluated extensively, and its silencing can occur via mutation, loss of heterozygosity (LOH), homozygous deletion, or promoter hypermethylation. In addition, p16 is a member of the cyclin dependent kinase inhibitor (CDKI) family of genes and causes cell cycle arrest at the G1/S phase. p16 inactivation can result in uncontrolled cell growth. Other genes involved in cell cycle regulation will be recognized and appreciated by one of skill in the art.

Other candidate members of genes that may serve as members of the gene panel include, but are not limited to genes involved in angiogenesis. Examples of genes involved in angiogenesis include but are not limited to TIMP-1, TIMP-2, TIMP-3, TIMP-4, VEGF-A, VEGF-B, VEGF-C, VEGF-D, VEGF-E, IL-8, TGFβ and TGFα to name a few. One of skill in the art can recognize and appreciate genes involved in angiogenesis.

Still other candidate member genes include, but are not limited to genes involved in DNA repair. Example of repair genes include, but are not limited to MGMT, BRCA1, BRCA2,hMLH1, hMSH1, hMLH6, and SHFM1 to name a few. One of skill in the art can recognize and appreciate DNA repair genes.

Additional candidate genes include, but are not limited to genes encoding receptors, growth factors and transcription factors to name a few. Some examples of a candidate for gene to serve on the panel include, but are not limited to, Hpp-1, sVEGFR-2 (sFLK-1), ESR1, IGFIR, IGFR, c-KIT, PDGFRα, HGFR, Grb2, bFGFR-2, FGFR-2, FGFR-3, PDEGF, RARBeta, and RASSF1A. Additional candidates include peptides containing epidermal growth factor like motifs, such as, but not limited to, NELL1 and NELL2.

In one embodiment, the panel of gene comprises a combination of at least 2, 3, 4 or 5 of the genes selected from the group consisting of Reprimo, p16, TIMP-3, MGMT Hpp-1, ESR1, RARβ and CHFR. In another embodiment, the panel of genes comprises the p16 and TIMP-3 genes. In yet another embodiment, the panel comprises ESR1 and RARβ.

The invention is not limited by the types of assays used to assess methylation status of the members of the gene or gene panel. Indeed, any assay that can be employed to determine the methylation status of the gene or gene panel should suffice for the purposes of the present invention. In general, assays are designed to assess the methylation status of individual genes, or portions thereof Examples of types of assays used to assess the methylation pattern include, but are not limited to, Southern blotting, single nucleotide primer extension, methylation-specific polymerase chain reaction (MSPCR), restriction landmark genomic scanning for methylation (RLGS-M) and CpG island microarray, single nucleotide primer extension (SNuPE), and combined bisulfite restriction analysis (COBRA). The COBRA technique is disclosed in Xiong, Z. and Laird, P., Nucleic Acids Research, 25(12): 2532-2534 (1997), which is incorporated by reference. In addition, methylation arrays may also be employed to determine the methylation status of a gene or panel of genes. Methylation arrays are disclosed in Beier V, et al., Adv Biochem Eng Biotechnol 1007; 104:1-11, which is incorporated by reference.

For example, a method for determining the methylation state of nucleic acids is described in U.S. Pat. No. 6,017,704 which is incorporated by reference. Determining the methylation state of the nucleic acid includes amplifying the nucleic acid by means of oligonucleotide primers that distinguishes between methylated and unmethylated nucleic acids.

Two or more markers, such as p 16 and TIMP-3 can also be screened simultaneously in a single amplification reaction to generate a low cost, reliable cancer-screening test for the likelihood that a polyp will recur. Methylation specific PCR (MSP) is disclosed in U.S. Pat. Nos. 5,786,146, 6,200,756, 6,017,704 and 6,265,171, each of which is incorporated by reference. Furthermore, a combination of DNA markers for CpG-rich regions of nucleic acid may be amplified in a single amplification reaction. The markers are multiplexed in a single amplification reaction, for example, by combining primers for more than one locus. In one embodiment, DNA from a normal tissue surrounding a polyp can be amplified with two or more different unlabeled or randomly labeled primer sets in the same amplification reaction. The reaction products can be separated on, for example, a denaturing polyacrylamide gel and subsequently exposed to film or stained with ethidium bromide for visualization and analysis.

By analyzing a panel of genes, there may be a greater probability of producing a more useful methylation profile for a subject. Multigene MSP may employ MSP primers for a plurality of markers, for example up to two, three, four, five or more different colorectal cancer marker, in a two-stage nested PCR amplification reaction. As in typical two stage primer PCR reactions, the primers used in the first PCR reaction are selected to amplify a larger portion of the target sequence than the primers of the second PCR reaction. The primers used in the first PCR reaction are generally referred to the DNA primers and the primers used in the second PCR reaction are the MSP primers. MSP primers generally comprise two sets of primers: methylated and unmethylated for each of the markers that are being assayed. Methods of multigene MSP are disclosed in U.S. Pat. No. 6,835, 541, which is incorporated by reference.

Detection of differential methylation can also be accomplished by contacting a nucleic acid sample with methylation-sensitive restriction endonucleases that cleave only unmethylated CpG sites under appropriate conditions and for an appropriate length of time to allow cleavage of unmethylated nucleic acid. The sample can also be contacted with isoschizomers of the methylation-sensitive restriction endonucleases that cleave both methylated and unmethylated CpG-sites under appropriate conditions and for an appropriate length of time to allow cleavage of methylated nucleic acid. Oligonucleotides are subsequently added to the nucleic acid sample under appropriate conditions and for an appropriate length of time to allow ligation of the added oligonucleotides to the cleaved nucleic acid. The ligated composition of nucleic acid from sample and oliogonucleotides can then be amplified by conventional methods, such as PCR, where the primers are complementary to the added oligonucleotides.

“Methylation-sensitive restriction endonuclease” are well known in the art and are generally considered to be is a restriction endonuclease that includes CG as part of its recognition site and has altered activity when the C is methylated as compared to when the C is not methylated. In one embodiment, the methylation-sensitive restriction endonuclease has inhibited activity when the C is methylated (e.g., Smal). Examples of methylation-sensitive restriction endonucleases include, but are not limited to, Sma I, BssHII, or HpaII, MspI, BSTUI, SacII, EagI, and NotI. Of course, these enzymes can be used alone or in combination with other enzymes. As used herein, an “isoschizomer” of a methylation-sensitive restriction endonuclease i a restriction endonuclease that recognizes the same recognition site as a methylation sensitive restriction endonuclease but cleaves both methylated and unmethylated CGs. Those of skill in the art can readily determine appropriate conditions for a restriction endonuclease to cleave a nucleic acid (see Sambrook et al., Molecular Cloning: A Laboratory Manual, Cold Spring Harbor Press, 1989).

The measure of the levels of methylation may contain a qualitative component, or it may be quantitative. For example, the methylation status of a gene or panel of genes may simply be considered, on the whole, as methylated or unmethylated, or the methylation status may be quantified as some numerical expression, such as a ratio or a percentage. Furthermore, the methylation status of each individual member of the gene or panel of genes may be assessed, or the methylation status of the gene or panel of genes, as a whole, may be assayed, determined or considered.

The methylation status of the subject may be assessed in vivo or in vitro, from a sample from the subject. The samples may or may not have been removed from their native environment. Thus, the portion of sample assayed need not be separated or removed from the rest of the sample or from a subject that may contain the sample. Of course, the sample may also be removed from its native environment. For example, the sample may be a tissue section. The tissue section may be, for example, a portion of the neoplasm that is being treated or it may be a portion of the surrounding normal tissue. Furthermore, the sample may be processed prior to being assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes freezing and/or preserving the sample prior to assaying.

Once the methylation status and level of the gene or panel of genes have been determined, these determinations can then be used to predict, indicate, or otherwise assess or predict the likelihood the abnormal condition, e.g., a polyp, will already be present, develop in the future, or recur in the future in the patient. As used herein, a subject in which the “condition recurred,” i.e., a progressor subject, is used to indicate that the abnormal condition recurred in the subject after successful ablative treatment. As used herein, “predict” means to provide an indicia of whether a particular abnormal condition will recur after treatment or if the abnormal condition will develop in subject. As used herein, indicate means to provide a basis to a health care practitioner whether a particular condition will recur in the subject.

To predict the development or recurrence of the abnormal condition, the methylation status or level of the test subject's gene or panel of genes may be compared to one or more progressor subjects, including, but not limited to a population of progressor subjects. Or the methylation status or level of the test subject's gene or panel of genes may be compared to one or more non-progressor subjects, including, but not limited to a population of non-progressor subjects. In addition, the methylation status or level of the gene or panel of genes in the test subject may be compared to his or her own previously assessed methylation status of the gene or panel of genes. In another embodiment, the methylation status or level of the gene or panel of genes in the test subject is compared to a normal methylation status or level of the gene or panel of genes.

“Normal methylation status or level” may be assessed by measuring the methylation status or level in a known healthy subject, including the same subject that is later screened or being diagnosed. Normal levels may also be assessed over a population of samples, where a population sample is intended to mean either multiple samples from a single subject or at least one sample from a multitude of subjects. Normal methylation levels of the gene or panel of genes, in terms of a population of samples, may or may not be categorized according to characteristics of the population including, but not limited to, sex, age, weight, ethnicity, geographic location, fasting state, state of pregnancy or post-pregnancy, menstrual cycle, general health of the subject, alcohol or drug consumption, caffeine or nicotine intake and circadian rhythms.

It will be appreciated by those of skill in the art that a baseline or normal level need not be established for each assay as the assay is performed but rather, baseline or normal levels can be established by referring to a form of stored information regarding a previously determined baseline methylation levels for a given gene or panel of genes, such as a baseline level established by any of the above-described methods. Such a form of stored information can include, for example, but is not limited to, a reference chart, listing or electronic file of population or individual data regarding “normal levels” (negative control) or polyp positive (including staged tumors) levels; a medical chart for the patient recording data from previous evaluations; a receiver-operator characteristic (ROC) curve; or any other source of data regarding baseline methylation levels that is useful for the patient to be diagnosed.

Further a methylation index (MI) may be established. A methylation index (MI) is defined as the number of genes which demonstrated altered methylation status (i.e., which exceed or fall below a previously determined methylation level cutoff) within a defined set of genes. For example, if there are four genes in a defined gene set and none of these four genes is methylated, the MI equals 0; if any one of the four are methylated, the MI equals 1; if any two of the four are methylated, the MI equals 2; if any three of the four are methylated, the MI equals 3; and if all four of these four genes are methylated, the MI equals 4 (i. e., the maximum possible MI for this gene set).

The difference between the methylation status or level of the test subject and normal methylation levels may be a relative or absolute quantity. Thus, “methylation level” or “methylation status” is used to connote any measure of the quantity of methylation of the gene or panel of genes. The level of methylation may be either abnormally high, or abnormally low, relative to a defined high or low threshold determined to be normal for a particular group of subjects. The difference in level of methylation between a subject and the reference methylation level may be equal to zero, indicating that the subject is or may be normal, or that there has been no change in levels of methylation since the previous assay.

The methylation levels and any differences that can be detected may simply be, for example, a measured fluorescent value, radiometric value, densitometric value, mass value etc., without any additional measurements or manipulations. Alternatively, the levels or differences may be expressed as a percentage or ratio of the measured value of the methylation levels to a measured value of another compound including, but not limited to, a standard or internal DNA standard, such as beta-actin. This percentage or ratio may be abnormally low, i. e., falling below a previously defined normal threshold methylation level; or this percentage or ratio may be abnormally high, i.e., exceeding a previously defined normal threshold methylation level. The difference may be negative, indicating a decrease in the amount of measured levels over normal value or from a previous measurement, and the difference may be positive, indicating an increase in the amount of measured methylation levels over normal values or from a previous measurement. The difference may also be expressed as a difference or ratio of the methylation levels to itself, measured at a different point in time. The difference may also be determined using in an algorithm, wherein the raw data is manipulated.

A difference between the test subject's methylation status between two time points is an indication that the test subject may or may have an increased likelihood of concurrent presence, future occurrence, or future recurrence of the abnormal condition in the subject. For example, a methylation status in the test subject at a first time point that is greater than the methylation status of the test subject at a second time point may indicate that there may be a lower likelihood of the concurrence, future occurrence, or recurrence of the abnormal condition in the subject, whereas the abnormal condition at time point one was predicted to be present, occur, or recur after treatment. Alternatively, a methylation status in the test subject that is lower at a first time point than the methylation status in the test subject at a second time point may indicate that the there is an increased likelihood that the abnormal condition will be present, occur, or recur in the subject, from the first time point. An inverse relationship, however, may also exist between the methylation status of the gene or panel of genes (or the difference thereof) and the subject's likelihood for an abnormal condition being present, developing in the future, or recurring in the future.

The present invention also provides methods of customizing a therapeutic regimen for a subject in need thereof, with the methods comprising determining the methylation status or level of a gene or panel of genes in a test subject and using the methylation status or level of the test subject to dictate an appropriate therapeutic regimen going forward or indicate the responsiveness of a particular therapeutic regimen going forward.

The present invention also provides methods of monitoring the progression of an abnormal condition in a subject, with the methods comprising determining the methylation status or level of a gene or panel of genes in a test subject at a first and second time point to determine a difference in methylation status or level of the gene or panel of genes in the subject over time. A difference in methylation status in the gene or panel of genes in the subject over time may be indicative of the occurrence, recurrence, or progression of the abnormal condition.

As used herein, the phrase “monitor the progression” is used to indicate that the abnormal condition in the subject is being periodically checked to determine if the abnormal condition is progression (worsening), regressing (improving), or remaining static (no detectable change) in the individual by assaying the methylation status or level in the subject using the methods of the present invention. The methods of monitoring may be used in conjunction with other monitoring methods or other treatments for the abnormal condition to monitor the efficacy of the treatment. Thus, “monitor the progression” is also intended to indicate assessing the efficacy of a treatment regimen by periodically assessing the methylation status of the gene or panel of genes and correlating any differences in methylation status in the subject over time with the progression, regression or stasis of the abnormal condition. Monitoring may include two time points from which a sample is taken, or it may include more time points, where any of the methylation status or level data at one particular time point from a given subject may be compared with the methylation status or level data in the same subject, respectively, at one or more other time points.

The present invention also provides methods of diagnosing a disease state in a subject suspected of having a disease, with the methods comprising determining the methylation status or level of a gene or panel of genes in a test subject and using the test subject's methylation status or level to indicate the presence of a disease state in the subject.

As used herein, the term “diagnose” means to confirm the results of other tests or to simply confirm suspicions that the subject may have an abnormal condition, such as cancer. A “test,” on the other hand, is used to indicate a screening method where the patient or the healthcare provider has no indication that the patient may, in fact, have an abnormal condition and may also be used to assess a patient's likelihood or probability of developing a disease or condition in the future. The methods of the present invention, therefore, may be used for diagnostic or screening purposes. Both diagnostic and testing can be used to “stage” the abnormal condition in a patient. As used herein, the term “stage” is used to indicate that the abnormal condition or obesity can be categorized, either arbitrarily or rationally, into distinct degrees of severity. The term “stage,” however, may or may not involve disease progression. The categorization may be based upon any quantitative characteristic or be based upon qualitative characteristics that can be separated. An example of staging includes but is not limited to the Tumor, Node, Metastasis System of the American Joint Committee on Cancer. For example, in stage T1 of colorectal cancer, the tumor has grown through the muscularis mucosa of the colon and extends into the submucosa. In stage T2, the cancer has grown through the submucosa, and extends into the muscularis propria. In stage T3, the cancer has grown completely through the muscularis propria into the subserosa, but not to any neighboring organs or tissues. And in stage T4, the cancer has spread completely through the wall of the colon or rectum into nearby tissues or organs. Other examples of staging systems include, but are not limited to, the Dukes system and the Astler-Coller system.

In one particular embodiment of the diagnostic methods, the present invention provides methods of assessing the probability of a subject having an abnormal condition, with the methods comprising determining a methylation status or level of at least one gene in grossly normal tissue of the subject and comparing the methylation status or level of the gene or genes in said subject to the normal methylation status or level of the at least one gene. As used herein, grossly normal tissue is used to indicate that the tissue from which the sample is taken appears normal upon gross inspection (i.e., by the naked eye). In other words, a technician or clinician who removes a sample or biopsy from the subject may remove the sample from what appears to be normal tissue. Once the grossly normal tissue is removed, DNA from the cells of the grossly normal tissue is isolated and the methylation status or level of a gene or panel of genes is determined in the cells' DNA that has been taken from the grossly normal tissue. The methylation status or level of the gene or panel of genes from the grossly normal tissue from the subject is then compared to the normal methylation status or level of the same gene or panel of genes to determine if any difference exists between the subject's status or level and previously defined normal status or level. A difference between the subject's methylation status or level and the normal methylation status or level of the gene or panel of genes indicates that the subject may have an altered probability of having or developing an abnormal condition elsewhere in the body. For example, the methylation status or level of a subject's rectum that is normal upon gross inspection can be compared to accepted normal methylation status or level. If a difference exists between the subject's methylation status or level in grossly normal rectum and the previously defined normal methylation status or level, this difference indicates that the subject may currently have, or develop in the future, an abnormal condition elsewhere in the remaining portion of the colon. These abnormal conditions that may be screened using grossly normal tissue from subjects include, but are not limited to, the abnormal conditions described herein.

The present invention also provides for kits for performing the methods described herein. Kits of the invention may comprise one or more containers containing one or more reagents useful in the practice of the present invention. Kits of the invention may comprise containers containing one or more buffers or buffer salts useful for practicing the methods of the invention. A kit of the invention may comprise a container containing a substrate for an enzyme, a set of primers and reagents for PCR, etc.

Kits of the invention may comprise one or more computer programs that may be used in practicing the methods of the invention. For example, a computer program may be provided that calculates a methylation status in a sample from results of the detecting levels of antibody bound to the biomarker gene product of interest. Such a computer program may be compatible with commercially available equipment, for example, with commercially available microarray or real-time PCR. Programs of the invention may take the output from microplate reader or realtime-PCR gels or readouts and prepare a calibration curve from the optical density observed in the wells, capillaries, or gels and compare these densitometric or other quantitative readings to the optical density or other quantitative readings in wells, capillaries, or gels with test samples.

### EXAMPLES

### Patient Selection

Rectal biopsies were obtained with informed consent from 53 patients that displayed colonic polyps. From patients with colonic polyps, biopsy was taken from polyp as well as from normal mucosa that was uninvolved with polyp or any other gross abnormality. Biopsy was also taken from normal rectum in patients not exhibiting any polyps or any other gross abnormality. Of the 81 patients displaying polyps, 31 were categorized as “progressors” as they displayed polyps at a follow-up colonoscopy, and 50 were characterized as “non-progressors” that did not display polyps at a follow-up colonoscopy.

### Gene Selection

Fifteen candidate genes were chosen based on known involvement or history of methylation in colon polyps or cancer and other tumor types, on previously reported preliminary findings in colon polyps, or due to their presumed or known roles in cellular functions related to cancer development. Specifically, Reprimo (the Greek word for “repress”) is a mediator of p53-mediated cell cycle arrest at the G2/M phase. (See Ohki, R., et al., J Biol Chem, 275:22627-22630 (2000), incorporated by reference). Reprimo is frequently methylated in a variety of human malignancies and is also induced by X-irradiation. (See Takahashi, T., et al., Int J Cancer, 115:503-510 (2005), incorporated by reference). (MGMT,) a DNA excision repair gene, is commonly methylated in cancer, (Eads, C. A., et al., Cancer Res, 61:3410-3418 (2001)), and promoter hypermethylation of MGMT has been correlated with a response to alkylating agents in brain tumors. (See Esteller, M., et al., N Engl J Med, 343:1350-1354, (2000), incorporated by reference). Tissue inhibitor of metalloproteinase-3 (TIMP-3) encodes a potent inhibitor of angiogenesis, and methylation of its promoter is associated with a poor prognosis in various cancers. (See Darnton, S. J., et al, Int J Cancer, 115: 351-358 (2005), incorporated by reference). p16 belongs to a family of cyclin-dependent kinase inhibitors that cause cell cycle arrest at the G1 phase. Methylation and subsequent lack of expression of p16 in various cancers are also associated with a poor prognosis. (See Brock, M. V., et al, Clin Cancer Res, 9:2912-2919 (2003), incorporated by reference). Methylation of RUNX-3 (runt-related transcription factor 3) is observed in at least esophageal cancer and is associated with progression from Barrett's esophagus with low-grade dysplasia to Barrett's adenocarcinoma. (See Schulmann, K. et al., Oncogene, 24:4138-4148 (2005)). Methylation of HPP1 (hyperplastic polyposis) is also correlated with Barrett's-associated neoplastic progression. (Schulmann, K. et al., Oncogene, 24:4138-4148 (2005)). Methylation of HPP1 is found in various cancers, (Schulmann, K. et al., Oncogene, 24:4138-4148 (2005)), and gastric and colon cancers (See Shibata, D. M., et al., Cancer Res, 62:5637-5640 (2002), Young, J., et al., Proc Natl Acad Sci U S A, 98:265-270 (2001) and Shibata, D., et al., Gastroenterology, 128:a-787 (2005), all of which are incorporated by reference). The exact function of HPP1 has not been determined, but it encodes an epidermal growth factor domain and is therefore thought to play a role in cell growth, maturation, and adhesion. (See Shibata, D. M., et al., Cancer Res, 62:5637-5640 (2002), Young, J., et al., Proc Natl Acad Sci U S A, 98:265-270 (2001)).

For our analysis of the predictive significance of the normal rectum, a set of methylation markers was used that was different from the markers used in the polyp evaluation. Markers used in polyps were specifically selected because they were not methylated in normal colonic mucosa. However, genes that are never methylated in normal mucosa will not be useful as markers in normal mucosa, since they will never show a positive finding. Thus, it was necessary to use markers that were differentially methylated between two sets of comparison groups: patients with and without index polyps; and patients with and without recurrent polyps. Relying upon Takahashi, T. et al, Int. J. Cancer 118(4):924-931 (2006) (incorporated by reference) four genes (SHP-1, DcR1, RARβ, and DcR2) were chosen because they were methylated about as frequently in matching normal colonic epithelium as in paired colorectal neoplastic lesions. SHP-1, DcR1, and RARβ were methylated in 80%, 75%, and 65% of normal mucosae from patients with concurrent colon cancer, respectively. Thus, the hypothesis was that these genes would be differentially methylated in normal rectal mucosa from patients without concurrent neoplasia or in patients not predisposed to developing future neoplastic lesions.

### DNA Treatment And Methylation-Specific PCR

Tumor samples were snap frozen on dry ice and stored at −80° C. After thawing, DNA was extracted from samples and treated with bisulfite prior to MSP. Briefly, DNA was extracted from all samples and treated with bisulfite to convert unmethylated cytosines to uracils prior to methylation-specific PCR (MSP) as described previously in Mori, Y., et al. Cancer Res. 64:2434-38 (2004), which is incorporated by reference. DNA methylation status and levels of the 4 candidate markers were determined with real-time quantitative MSP using the ABI 7900 HT Sequence Detection (Taqman) System, as described previously in Sato F., et al., Cancer Res. 62:6820-22 (2002), which is incorporated by reference. Primers and probes for quantitative MSP of (SHP-1, DcR1, RARβ, and DcR2) are disclosed in Takahashi, T. et al, Int'l J. Cancer, 118(4): 924-931 (2005), which is incorporated by reference.

The Sodium Bisulfite Conversion of DNA was performed using the EpiTect BiSulfite Kit, available from Qiagen, according to the manufacturer's suggested protocol. Briefly, DNA was thawed and dissolve by adding 800 μl RNase-free water to each aliquot. The dissolved DNA was vortexed until the Bisulfite Mix was completely dissolved. On occasion, it was necessary to heat the water/DNA mixture to about 60° C. to aid in dissolving of the DNA. Bisulfite reactions were prepared in 200 μl PCR tubes according to Table I (each component was added in the order listed).

TABLE I
Bisulfite Reaction Components

| Component | Volume per Reaction (μL) |
|---|---|
| DNA solution (1 ng-2 μg) | Variable* |
| | (maximum 20) |
| RNase-free water | Variable* |
| Bisulfite Mix (dissolved) | 85 |
| DNA Protect Buffer | 35 |
| Total volume | 140 |

*The combined volume of DNA solution and RNase-free water must total 20 μl.

After mixing, the PCR tubes are stored at room temperature. Next, the bisulfite DNA conversion was performed using a thermal cycler that was programmed according to the parameters in Table II.

TABLE II
Bisulfite Conversion Thermal Cycler Conditions

| Step | Time | Temperature |
|---|---|---|
| Denaturation | 5 Min | 99° C. |
| Incubation | 25 Min | 60° C. |
| Denaturation | 5 Min | 99° C. |
| Incubation | 85 Min | 60° C. |
| Denaturation | 5 Min | 99° C. |
| Incubation | 175 Min | 60° C. |
| Hold | Indefinite | 20° C. |

Once the bisulfite conversion was complete, the PCR tubes were centrifuged and transferred to clean 1.5 ml microcentrifuge tubes. 560 μl of freshly prepared Buffer BL (containing 10 μg/ml carrier RNA) was then added and mixed by vortexing and centrifugation. The EpiTect spin columns were placed in a and collection tube in a suitable rack and the mixture was transferred into the EpiTect spin column. The columns were centrifuged at maximum speed for about 1 minute and the flow-through was discarded. The spin columns were placed back into the collection tubes and 500 μl Buffer BW (wash buffer) was to the spin columns. Again, the spin columns were centrifuged at maximum speed for about 1 minute, and the flow-through was discarded. The spin columns were placed back into the collection tubes.

Next, 500 μl of Buffer BD (desulfonation buffer) was added to each spin column, and the columns were incubated for about 15 minutes at room temperature. After incubation, the columns were centrifuged at maximum speed for about 1 minute. The flow-through was discarded, and the columns were placed back into the collection tubes.

500 μl Buffer BW was added to the columns and the columns were centrifuged at maximum speed for about 1 min. The flow-through was discarded, and the spin columns were placed back into the collection tube. This washing step was repeated at lease one more time.

After repeated washing, the spin columns were placed into new 2 ml collection tube, and the columns were centrifuged at maximum speed for about 1 to 5 minutes to remove any residual liquids. Finally, the spin columns were placed into clean 1.5 ml microcentrifuge tubes and 20 μl of Buffer EB was to the center of the membrane in the spin column. The purified DNA was then eluted by centrifugation for about 1 minute at approximately 15,000×g (12,000 rpm).

DNA methylation status and levels of 15 genes were determined with real-time quantitative MSP using the ABI 7900 HT Sequence Detection (Taqman) System, as described previously in Sato F., et al., Cancer Res. 62:6820-22 (2002), which is incorporated by reference. Primers and probes for quantitative MSP of p16, TIMP-3, APC, MGMT, RIZ1, HPP1, ACTB and p14 are disclosed in Sato, F., et al., Cancer Res. 62:6820-22 (2002), Sato, F., et al, Cancer Res. 62:1148-51 (2002) and Eads, C., et al, Cancer Res. 61:3410-18 (2001), which are incorporated by reference.

TABLE III
Forward and Reverse Primers

| | | |
|---|---|---|
| Reprimo | Frwd | 5′-CGC GTC GGA AGG GGT C-3′ |
| | | (SEQ ID NO. 1) |
| | Rev | 5′-ACT CGT TCC CGA CGC TCG-3′ |
| | | (SEQ ID NO. 2) |
| p16 | Frwd | 5′-TGGAATTTTCGGTTGATTGGTT-3′ |
| | | (SEQ ID NO. 3) |
| | Rev | 5′-AACAACGTCCGCACCTCCT-3′ |
| | | (SEQ ID NO. 4) |
| TIMP-3 | Frwd | 5′-GCGTCGGAGGTTAAGGTTGTT-3′ |
| | | (SEQ ID NO. 5) |
| | Rev | 5′-CTCTCCAAAATTACCGTACGCG-3′ |
| | | (SEQ ID NO. 6) |
| RUNX-3 | Frwd | 5′-GGGTTTTGGCGAGTAGTGGTC-3′ |
| | | (SEQ ID NO. 7) |
| | Rev | 5′-ACGACCGACGCGAACG-3′ |
| | | (SEQ ID NO. 8) |
| MGMT | Frwd | 5′-CTAACGTATAACGAAAATCGTAACAACC-3′ |
| | | (SEQ ID NO. 9) |
| | Rev | 5′-AGTATGAAGGGTAGGAAGAATTCGG-3′ |
| | | (SEQ ID NO. 10) |
| Hpp-1 | Frwd | 5′-GTTATCGTCGTCGTCGTTTTTGTTGTC-3′ |
| | | (SEQ ID NO. 11) |
| | Rev | 5′-GACTTCCGAAAAACACAAAATCG-3′ |
| | | (SEQ ID NO. 12) |
| β-Actin | Frwd | 5′-TGGTGATGGAGGAGGTTTAGTAAGT-3′ |
| | | (SEQ ID NO. 13) |
| | Rev | 5′-AACCAATAAAACCTACTCCTCCCTTAA-3′ |
| | | (SEQ ID NO. 14) |

TABLE IV
Methylation-Specific PCR Probes

| | |
|---|---|
| Reprimo | 6FAM-TTA AAA CTT AAC GAA ACT AAA CCA ACC |
| | CGA CCG T-TAMRA |
| | (SEQ ID NO. 15) |
| p16 | 6FAM-FAM-ACCCGACCCCGAACCGCG-TAMRA |
| | (SEQ ID NO. 16) |
| TIMP-3 | 6FAM-AACTCGCTCGCCCGCCGAA-TAMRA |
| | (SEQ ID NO. 17) |
| MGMT | 6FAM-CCTTACCTCTAAATACCAACCCCAAACCCG-TAMRA |
| | (SEQ ID NO. 18) |
| RUNX-3 | 6FAM-CGTTTTGAGGTTCGGGTTTCGTCGTT6-TAMRA |
| | (SEQ ID NO. 19) |
| Hpp-1 | 6FAM-CCGAACAACGAACTACTAAACATCCCGCG-TAMRA |
| | (SEQ ID NO. 20) |
| β-Actin | 6VIC-ACCACCACCCAACACACAATAACAAACACA-TAMRA |
| | (SEQ ID NO. 21) |

A normalized methylation value (NMV) reflecting the percentage of DNA methylated for the gene of interest (GoI), was defined as follows: NMV=(GoI-S/GoI-FM)/(ACTB-S/ACTB-FM)*100, where GoI-S and GoI-FM represented GoI methylation levels in the Sample and Fully Methylated DNAs, respectively, while ACTB-S and ACTB-FM corresponds to β-Actin in the sample and Fully Methylated (FM) DNAs, respectively.

### Statistical Analysis

Single-parameter parametric (Student's t-test) and nonparametric (Mann-Whitney U test) testing was used to test the selected genes as markers for index adenoma. The software package was Statistica (version 6.1; StatSoft, Inc., Tulsa, Okla.). Surprisingly, the Mann-Whitney calculations revealed a statistically significant finding of retinoic acid receptor beta (RAR-β) was methylated significantly more frequently in the normal rectum of patients without polyps than those with polyps (p=0.032146, sigma-restricted parameterization, general regression model).

### Markers For Adenoma Recurrence

The methylation status of colon adenomagenic genes TAC1, SST, and NELL1 were studied, along with the ESR1, HPP1, MGMT, MLH1, p14, p16, RARβ, and TIMP3 genes. In addition, 2 clinical parameters, patient age and maximum polyp size at the time of index polypectomy, were also measured. Quantitative methylation levels were assessed in 81 index polyps using quantitative methylation-specific PCR (qMSP). The marker genes were selected based on known molecular abnormalites or methylation in colon polyps, colon cancer, or other tumor types, on our own reported preliminary findings in colon polyps, or on their known roles in cellular functions related to cancer development.

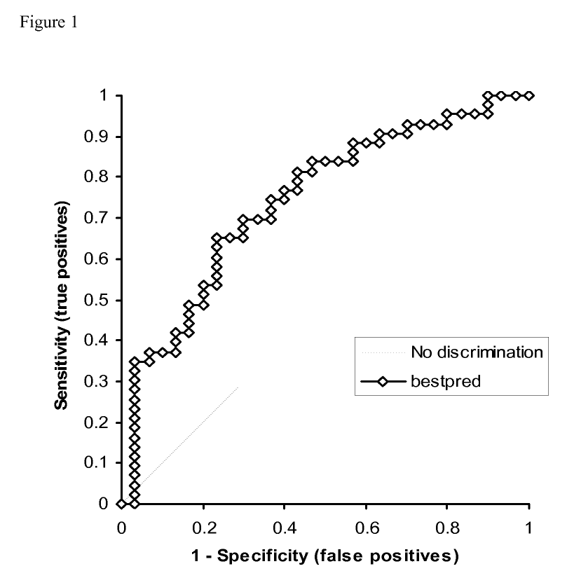

FIG. 1 is a graph of dataset of methylation status of APC, MGMT, MLH1, NELL1 and RARβ and demonstrates the best AUROC using linear discriminant analysis and leave-one-out crossvalidation vs. polyp recurrence. Methylation of MLH1, NELL1, and RARβ correlated inversely with adenoma recurrence. A cutoff value of 5% methylation was set prior statistical analysis to define positive vs. negative methylation in the index sample. ROC curve analyses were performed using Analyse-It +Clinical Laboratory 1.71. AUROC=0.7434.

### Markers For Concurrent Polyp Based Upon Methylation Status Of An Index Polyp

In another analysis, the methylation status of an index polyp was examined to determine its value in predicting a concurrent polyp elsewhere in the colon. Results of the correlations are displayed in Table V.

TABLE V

| | Mean no | Mean yes | t-value | p |
|---|---|---|---|---|
| Age | 66.20000 | 68.61290 | −1.24466 | 0.216936 |
| APC | 0.07144 | 0.09618 | −0.89183 | 0.375191 |
| ESR1 | 0.18739 | 0.19109 | −0.11857 | 0.905916 |
| HPP1 | 0.19293 | 0.17948 | 0.35041 | 0.726964 |
| MGMT | 0.04277 | 0.04282 | −0.00350 | 0.997214 |
| MLH1 | 0.00087 | 0.00066 | 0.92430 | 0.358147 |
| NELL1 | 0.33878 | 0.10195 | 1.16405 | 0.247908 |
| P14 | 0.04608 | 0.06019 | −0.85071 | 0.397503 |
| P16 | 0.00559 | 0.00880 | −0.89223 | 0.374981 |
| RAR Beta | 0.40903 | 0.22281 | 2.07131 | 0.041594 |
| SST | 0.30547 | 0.29890 | 0.15132 | 0.880107 |
| TAC1 | 0.16778 | 0.13083 | 0.97679 | 0.331657 |
| TIMP3 | 0.02949 | 0.01672 | 1.15693 | 0.250789 |
| Biggest polyp's size | 1.03273 | 0.80470 | 1.20571 | 0.231528 |

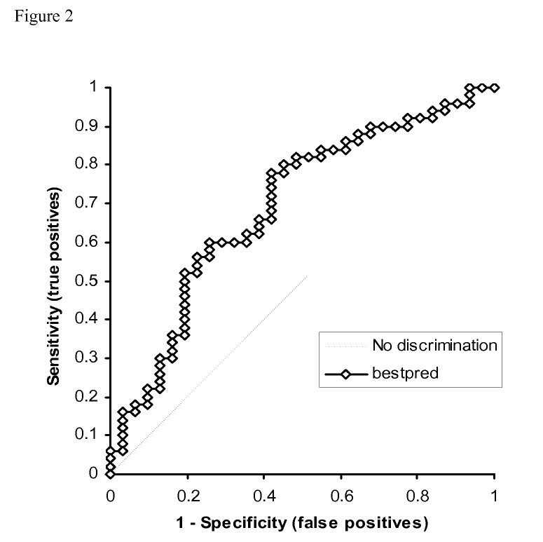

FIG. 2 depicts the ROC curve based on dataset composed of age, APC, MLH1, p16, RARβ, and biggest polyp size. This dataset exhibited the best AUROC using linear discriminant analysis and leave-one-out crossvalidation vs. the presence of a concurrent adenoma at the same time as the index polypectomy. Methylation of MLH1, RARβ and biggest polyp size correlated inversely with adenoma concurrence. A cutoff value of 5% methylation was set prior to statistical analysis define positive vs. negative methylation in the index sample. ROC curve analyses were performed using Analyse-It+Clinical Laboratory 1.71. AUROC=0.6929.

### Markers For Concurrent Polyp Prediction From Grossly Normal Rectal Tissue

In another study, the methylation status of 13 genes (APC, CDH1, ESR1, HIN1, HPP1, MGMT, NELL1, p14, p15, RARβ, SST, TAC1, and TIMP-3) in each of 86 normal rectum samples (58 from subjects with concurrent colorectal adenomas, 52 without concurrent adenomas). Primer and probe sequences are listed in Table VI.

TABLE VI

| Target | Sequence | |
|---|---|---|
| gene | description | Sequence |
| 3OST2 | Dual-labeled probe | 5′-\56-FAM\CGAACAACCGAACGACTCGAACGCT\36-TAMTph\-3′ |
| CDH1 3 | Forward primer | 5′-TCGCGGGGTTCGTTTTTCGC-3′ |
| CDH1 3 | Reverse primer | 5′-GACGTTTTCATTCATACACGCG-3′ |
| HPP 1 | Dual-labeled probe | 5′-\56-FAM\CCGAACAACGAACTACTAAACATCCCGCG\36-TAMTph\-3′ |
| HPP 1 | Forward primer | 5′-GTTATCGTCGTCGTTTTTGTTGTC-3′ |
| HPP1 | Reverse primer | 5′-GACTTCCGAAAAACACAAAATCG-3′ |
| MGMT | Dual-labeled probe | 5′-\56-FAM\CCTTACCTCTAAATACCAACCCCAAACCCG\36-TAMTph\-3′ |
| MGMT | Forward primer | 5′-AGTATGAAGGGTAGGAAGAATTCGG-3′ |
| MGMT | Reverse primer | 5′-CTAACGTATAACGAAAATCGTAACAACC-3′ |
| MLH1 | Dual-labeled probe | 6FAM-CGCGACGTCAAACGCCACTACG-TAMRA |
| MLH1 | Forward primer | 5′-CGTTATATATCGTTCGTAGTATTCGTGTTT-3′ |
| MLH1 | Reverse primer | 5′-CTATCGCCGCCTCATCGT-3′ |
| CRBP1 | Forward primer | 5′-TTG GGA ATT TAG TTG TCG TCG TTT C-3′ |
| CRBP1 | Reverse primer | 5′-AAA CAA CGA CTA CCG ATA CTA CGC G-3′ |
| P16 | Dual-labeled probe | 5′-\5Cy5\ACCCGACCCCGAACCGCG\3BHQ_2\-3′ |
| P16 | Forward primer | 5′-TGGAATTTTCGGTTGATTGGTT-3′ |
| P16 | Reverse primer | 5′-AACAACGTCCGCACCTCCT-3′ |
| RASS1FA | Dual-labeled probe | 5′-\56-FAM\CCGACATAACCCGATTAAACCCGTACTTCG\36-TAMTph\-3′ |
| RASS1FA | Forward primer | 5′-CGATACCCCGCGCGA-3′ |
| RASS1FA | Reverse primer | 5′-GTGGTTTCGTTCGGTTCGC-3′ |
| RIZ1 | Dual-labeled probe | 5′-\56-FAM\CGACGGCGTAGGGTTAAGGGTCG\36-TAMTph\-3′ |
| RIZ1 | Forward primer | 5′-GGATTCGCGGTGATTTACGA-3′ |
| RIZ1 | Reverse primer | 5′-CTACGAAACTAAAAAACTCCGAAACC-3′ |
| RUNX3 | Dual-labeled probe | 5′-\56-FAM\CGTTTTGAGGTTCGGGTTTCGTCGTT\36-TAMTph\-3′ |
| RUNX3 | Forward primer | 5′-gggTTTtggcgagtagtggTc-3′ |
| RUNX3 | Reverse primer | 5′-GAAAACGACCGACGCGAACG-3′ |
| SOCS1 | Dual-labeled probe | 5′-\56-FAM\TTAGAAGAGAGGGAAATAGGGTCGAAGCGG\36-TAMTph\-3′ |
| SOCS1 | Forward primer | 5′-ttcgcgtgtatttttaggtcggtc/gttgtaggatggggtcgcggtcgc-3′ |
| SOCS1 | Reverse primer | 5′-gttgtaggatggggtcgcggtcgc/ctactaaccaaactaaaatccaca-3′ |
| CDH1 | Dual-labeled probe | 5′-AATTTTAGGTTAGAGGGTTATCGCGT-3′ |
| CDH1 | Forward primer | 5′-\56-FAM\CGCCCACCCGACCTCGCAT\36-TAMTph\-3′ |
| CDH1 | Reverse primer | 5′-TCCCCAAAACGAAACTAACGAC-3′ |
| ESR | Dual-labeled probe | 5′-\56-FAM\CGATAAAACCGAACGACCCGACGA\36-TAMTph\-3′ |
| ESR | Forward primer | 5′-GGCGTTCGTTTTGGGATTG-3′ |
| ESR | Reverse primer | 5′-GCCGACACGCGAACTCTAA-3′ |
| APC | Dual-labeled probe | 5′-\5TexRd-XN\CCCGTCGAAAACCCGCCGATTA\3BHQ_2\-3′ |
| APC | Forward primer | 5′-GAACCAAAACGCTCCCCAT-3′ |
| APC | Reverse primer | 5′-TTATATGTCGGTTACGTGCGTTTATAT-3′ |
| CHFR | Forward primer | 5′-GTAATGTTTTTTGATAGCGGC-3′ |
| CHFR | Reverse primer | 5′-AATCCCCCTTCGCCG-3′ |
| HIN1 | Dual-labeled probe | 6FAM-acttcctactacgaccgacgaacc-TAMRA |
| HIN1 | Forward primer | 5′-tagggaagggggtacgggttt-3′ |
| HIN1 | Reverse primer | 5′-cgctcacgaccgtaccctaa-3′ |
| P14 | Dual-labeled probe | 5′-\56-FAM\CGAAAACCCTCACTCGCGACGAACCGC\36-TAMTph\-3′ |
| P14 | Forward primer | 5′-GGTGATTTTTCGGATTCGGC-3′ |
| P14 | Reverse primer | 5′-CACTCCCCCGTAAACCGCGA-3′ |
| THBS1 | Dual-labeled probe | 5′-\56-FAM\ACGCCGCGCTCACCTCCCT\36-TAMTph\-3′ |
| THBS1 | Forward primer | 5′-CGACGCACCAACCTACCG-3′ |
| THBS1 | Reverse primer | 5′-GTTTTGAGTTGGTTTTACGTTCGTT-3′ |
| DCR1 | Dual-labeled probe | 5′-TGATTAGAGATGTAAGGGGTGAAGGAGC |
| DCR1 | Forward primer | 5′-TTACGCGTACGAATTTAGTTAAC-3′ |
| DCR1 | Reverse primer | 5′-TTTTACGCGTACGAATTTAGTTAAC-3′ |
| RAR-bata | Dual-labeled probe | 5′-TCGGAACGTATTCGGAAGGTTTTTTGTAAGT-3′ |
| RAR-bata | Forward primer | 5′-CGAGAACGCGAGCGATTC-3′ |
| RAR-bata | Reverse primer | 5′-CAAACTTACTCGACCAATCCAACC-3′ |
| SHP1 | Dual-labeled probe | 5′-tcggtatttagtaggatttattcgatgatagttgttatcgt-3′ |
| SHP1 | Forward primer | 5′-ggtatgtgaacgttattatagtatagc-3′ |
| SHP1 | Reverse primer | 5′-ggttagggagggttgc-3′ |
| TIMP3 | Dual-labeled probe | 5′-\56-FAM\AACTCGCTCGCCCGCCGAA\36-TAMTph\-3′ |
| TIMP3 | Forward primer | 5′-CTCTCCAAAATTACCGTACGCG-3′ |
| TIMP3 | Reverse primer | 5′-GCGTCGGAGGTTAAGGTTGTT-3′ |
| TGFBR2 | Dual-labeled probe | 5′-\56-FAM\CACGAACGACGCCTTCCCGAA\36-TAMTph\-3′ |
| TGFBR2 | Forward primer | 5′-CAAACCCCGCTACTCGTCAT-3′ |
| TGFBR2 | Reverse primer | 5′-GCGCGGAGCGTAGTTAGG-3′ |
| BACT | Dual-labeled probe | 5′-\5HEX\ACCACCACCCAACACACAATAACAAACACA\3BHQ_1\-3′ |
| BACT | Forward primer | 5′-TGGTGATGGAGGAGGTTTAGTAAGT-3′ |
| BACT | Reverse primer | 5′-AACCAATAAAACCTACTCCTCCCTTAA-3′ |
| CD9 | Dual-labeled probe | 5′-\56-fam\acaaccactccctaccacttttaccgcgaactta\36-tamtph\-3′ |
| CD9 | Forward primer | 5′-GGGGGAATCGGAAGGGC-3′ |
| CD9 | Reverse primer | 5′-ACCCACTCCTTCTTCAAACCG-3′ |
| p15 | Dual-labeled probe | 5′-AGGAAGGAGAGAGTGCGTCG-3′ |
| p15 | Forward primer | 5′-\56-FAM\TTAACGACACTCTTCCCTTCTTTCCCACG\36-TAMTph\-3′ |
| p15 | Reverse primer | 5′-CGAATAATCCACCGTTAACCG-3′ |

Methylation levels of each of these genes, patient age and the presence/absence of one or more concurrent polyps found on colonoscopy performed at the time of the rectal biopsy were correlated using Student's t-testing. Results of these correlations are displayed in Table VII, below.

TABLE VII

| | Yes | No | t-value | p |
|---|---|---|---|---|
| Age | 66.72340 | 63.71795 | 1.20729 | 0.230708 |
| APC | 0.01199 | 0.03045 | −1.64403 | 0.103908 |
| CDH1 | 0.03536 | 0.03929 | −0.43691 | 0.663294 |
| ESR1 | 0.14038 | 0.19880 | −1.57682 | 0.118597 |
| HIN1 | 0.02245 | 0.02381 | −0.30678 | 0.759768 |
| HPP1 | 0.03629 | 0.04927 | −0.92579 | 0.357209 |
| MGMT | 0.04224 | 0.05771 | −2.48845 | 0.014805 |
| NELL1 | 0.04770 | 0.05623 | −0.37605 | 0.707826 |
| P14 | 0.01393 | 0.02636 | −2.03715 | 0.044784 |
| P15 | 0.00963 | 0.01242 | −1.31308 | 0.192732 |
| RARb | 0.79019 | 1.27761 | −2.32344 | 0.022572 |
| SST | 0.50775 | 0.57070 | −0.50177 | 0.617143 |
| TAC1 | 0.12200 | 0.16737 | −1.54491 | 0.126128 |
| TIMP3 | 0.01740 | 0.01644 | 0.24163 | 0.809659 |

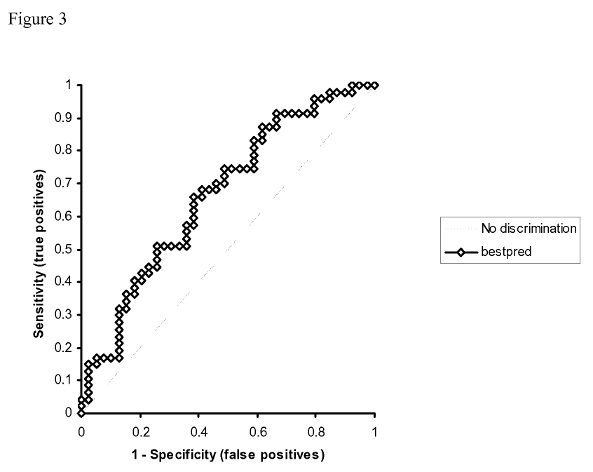

FIG. 3 is a ROC curve based on dataset composed of age, APC, NELL1, p14, and a methylation index (composed of APC, ESR1, HPP1, MGMT, p14, p15, RAR_{.}³¹ , and TAC1). The dataset exhibited the best AUROC using linear discriminant analysis and leave-one-out crossvalidation vs. the presence of a concurrent adenoma at the time of index polypectomy. A cutoff value of 5% methylation was set a priori to define positive vs. negative methylation in the index sample. ROC curve analyses were performed using Analyse-It+Clinical Laboratory 1.71. AUROC=0.6661.

### Smoking As A Predictive Parameter

The same dataset as in the previous example was examined, except that patients under the age of 50 and patients whose smoking status was uncertain were eliminated from analysis. Smokers were defined as active smokers or smokers with at least 20 pack-years of smoking history. Non-smokers were defined as patients with no history of smoking. The results in Table VIII indicate that the chances of demethylation of certain genes is correlated with smoking status rather than age. Indeed, there was no significant difference between methylation status as a function of age (data not shown).

TABLE VIII

| | | ESR1 | MGMT | P15 | RAR β | SST | TAC1 |
|---|---|---|---|---|---|---|---|
| Average | nonsmokers | 23.18% | 5.57% | 1.34% | 106.21% | 62.54% | 18.07% |
| methylation | smokers | 14.63% | 3.91% | 0.83% | 77.31% | 40.74% | 12.66% |
| percentage for | | | | | | | |
| p value for 50+ | | 0.023614 | 0.017318 | 0.023435 | 0.1783 | 0.089988 | 0.069562 |
| p value for 55+ | | 0.019902 | 0.010312 | 0.005858 | 0.066442 | 0.094772 | 0.028982 |
| p value for 60+ | | 0.016256 | 0.03245 | 0.003162 | 0.009284 | 0.082969 | 0.022149 |
| p value for 65+ | | 0.002364 | 0.017354 | 0.007496 | 0.001061 | 0.002358 | 0.014907 |
| p value for 70+ | | 0.009882 | 0.033451 | 0.005662 | 0.003613 | 0.009632 | 0.048961 |
| p value for 75+ | | 0.049159 | 0.188435 | 0.006617 | 0.053028 | 0.04266 | 0.075533 |
| p value for 80+ | | 0.136957 | 0.09963 | 0.016398 | 0.16078 | 0.075363 | 0.128152 |

| odds ratio of having a polyps smokers vs. non smokers | Age group |
|---|---|
| 1.01434426 | Over 50 |
| 1.03703704 | Over 55 |
| 1.06140351 | Over 60 |
| 1.00740741 | Over 65 |
| 1.15942029 | Over 70 |
| 1.73333333 | Over 75 |
| 1.71428571 | Over 80 |

Patient population = 92 (42 without polyps, 50 with polyps; 30 non-smokers and 62 smokers)

Based upon the p-values and the methylation levels of marker genes in otherwise gross normal rectal tissue, a decrease in methylation of marker genes was shown to be an indicator of the patient having a concurrent polyp elsewhere in the colon, independent of age group. In addition, a decrease in methylation of marker genes was shown to be an indicator of the patient having a history of smoking, regardless of age group.

What is claimed is:

1. A method for assessing the probability of the recurrence of an abnormal condition in a subject, said method comprising
a) determining a methylation status of at least one gene in the subject; and
b) comparing the methylation status of said at least one gene in said subject to the normal methylation status of said at least one gene;
wherein a difference between the methylation status of said at least one gene in said subject and the normal methylation status of said at least one gene indicates the altered probability of the recurrence of the abnormal condition in the subject.

2. The method of claim 1, wherein said abnormal condition is neoplastic growth.

3. The method of claim 2, wherein said abnormal condition is colon polyp formation.

4. The method of claim 3, wherein said altered probability is an increased probability of the recurrence of the colon polyps.

5. The method of claim 4, wherein said at least one gene is the adenomatous polyposis coli (APC) gene.

6. The method of claim 5, wherein said difference that indicates an increased probability of recurring colon polyps is positive.

7. The method of claim 6, wherein said determining said methylation status comprises using an assay selected from the group consisting of Southern blotting, single nucleotide primer extension, methylation-specific polymerase chain reaction (MSP), restriction landmark genomic scanning for methylation (RLGS-M), CpG island microarray, SNUPE, and COBRA.

8. The method of claim 1, wherein the methylation status of a panel of genes is determined and compared to the normal methylation status of said panel of genes.

9. The method of claim 8, wherein said panel comprises two or more genes.

10. The method of claim 9, wherein said panel comprises at least 3, 4 or 5 genes.

11. The method of claim 10, wherein said panel comprises at least 5 genes.

12. The method of claim 11, wherein said panel comprises adenomatous polyposis coli (APC) gene, O ⁶-methylguanine-DNA methyltransferase (MGMT) gene, mutL homolog 1 (MLH1) gene, nel-like type 1 (NELL 1) gene and retinoic acid receptor-beta (RARE) gene.

13. A method of monitoring the recurrence of an abnormal condition in a subject, said method comprising
a) determining a methylation status of at least one gene in said subject at a first and second time point; and
b) determining a difference between said methylation state at said first and second time points to assess a change of methylation state over time;
wherein said difference over time is indicative of a change in the subject's probability of the recurrence of said abnormal condition.

14. A method of monitoring the development of an abnormal condition in a subject, said method comprising
a) determining a methylation status of at least one gene in said subject at a first and second time point; and
b) determining a difference between said methylation status at said first and second time points to assess a change of methylation status over time;
wherein said difference over time is indicative of a change in the subject's probability of developing said abnormal condition.

15. A method for assessing the probability of a subject having an abnormal condition, said method comprising
a) determining a methylation status of at least one gene in gross normal tissue of the subject; and
b) comparing the methylation status of said at least one gene in said subject to the normal methylation status of said at least one gene;
wherein a difference between the methylation status of said at least one gene in said gross normal tissue of said subject and the normal methylation status of said at least one gene indicates that the subject has an altered probability of having said abnormal condition.

16. The method of claim 15, wherein said gross normal tissue is rectal tissue.

17. The method of claim 16, wherein said abnormal condition is neoplastic growth.

18. The method of claim 16, wherein said abnormal condition is colon polyp formation.

19. The method of claim 18, wherein said altered probability is an increased probability of having said colon polyps.

20. The method of claim 19, wherein said at least one gene is the adenomatous polyposis coli (APC) gene.

21. The method of claim 19, wherein said difference that indicates an increased probability of having said colon polyps is negative.

22. The method of claim 20, wherein said determining said methylation status comprises using an assay selected from the group consisting of Southern blotting, single nucleotide primer extension, methylation-specific polymerase chain reaction (MSPCR), restriction landmark genomic scanning for methylation (RLGS-M), CpG island microarray, SNUPE, and COBRA.

23. The method of claim 15, wherein the methylation status of a panel of genes is determined and compared to the normal methylation status of said panel of genes.

24. The method of claim 23, wherein said panel comprises two or more genes.

25. The method of claim 24, wherein said panel comprises at least 3, 4 or 5 genes.

26. The method of claim 25, wherein said panel comprises at least 5 genes.

27. The method of claim 26, wherein said panel comprises adenomatous polyposis coli (APC) gene, O⁶-methylguanine-DNA methyltransferase (MGMT) gene, mutL homolog 1 (MLH1) gene, nel-like type 1 (NELL 1) gene and retinoic acid receptor-beta (RARE) gene.


SEQUENCE LISTING (95 sequences)

SEQ ID NO: 1 - DNA, 16 bp, artificial
  Primer Reprimo
cgcgtcggaaggggtc

SEQ ID NO: 2 - DNA, 18 bp, artificial
  Primer
actcgttcccgacgctcg

SEQ ID NO: 3 - DNA, 22 bp, artificial
  primer
tggaattttcggttgattggtt

SEQ ID NO: 4 - DNA, 19 bp, artificial
  primer
aacaacgtccgcacctcct

SEQ ID NO: 5 - DNA, 21 bp, artificial
  primer
gcgtcggaggttaaggttgtt

SEQ ID NO: 6 - DNA, 22 bp, artificial
  primer
ctctccaaaattaccgtacgcg

SEQ ID NO: 7 - DNA, 21 bp, artificial
  primer
gggttttggcgagtagtggtc

SEQ ID NO: 8 - DNA, 16 bp, artificial
  primer
acgaccgacgcgaacg

SEQ ID NO: 9 - DNA, 28 bp, artificial
  primer
ctaacgtataacgaaaatcgtaacaacc

SEQ ID NO: 10 - DNA, 25 bp, artificial
  primer
agtatgaagggtaggaagaattcgg

SEQ ID NO: 11 - DNA, 27 bp, artificial
  primer
gttatcgtcgtcgtcgtttttgttgtc

SEQ ID NO: 12 - DNA, 23 bp, artificial
  primer
gacttccgaaaaacacaaaatcg

SEQ ID NO: 13 - DNA, 25 bp, artificial
  primer
tggtgatggaggaggtttagtaagt

SEQ ID NO: 14 - DNA, 27 bp, artificial
  primer
aaccaataaaacctactcctcccttaa

SEQ ID NO: 15 - DNA, 34 bp, artificial
  primer
ttaaaacttaacgaaactaaaccaacccgaccgt

SEQ ID NO: 16 - DNA, 18 bp, artificial
  primer
acccgaccccgaaccgcg

SEQ ID NO: 17 - DNA, 19 bp, artificial
  primer
aactcgctcgcccgccgaa

SEQ ID NO: 18 - DNA, 30 bp, artificial
  primer
ccttacctctaaataccaaccccaaacccg

SEQ ID NO: 19 - DNA, 26 bp, artificial
  primer
cgttttgaggttcgggtttcgtcgtt

SEQ ID NO: 20 - DNA, 29 bp, artificial
  primer
ccgaacaacgaactactaaacatcccgcg

SEQ ID NO: 21 - DNA, 30 bp, artificial
  primer
accaccacccaacacacaataacaaacaca

SEQ ID NO: 22 - DNA, 25 bp, artificial
  primer
cgaacaaccgaacgactcgaacgct

SEQ ID NO: 23 - DNA, 20 bp, artificial
  primer
tcgcggggttcgtttttcgc

SEQ ID NO: 24 - DNA, 22 bp, artificial
  primer
gacgttttcattcatacacgcg

SEQ ID NO: 25 - DNA, 29 bp, artificial
  primer
ccgaacaacgaactactaaacatcccgcg

SEQ ID NO: 26 - DNA, 24 bp, artificial
  primer
gttatcgtcgtcgtttttgttgtc

SEQ ID NO: 27 - DNA, 23 bp, artificial
  primer
gacttccgaaaaacacaaaatcg

SEQ ID NO: 28 - DNA, 30 bp, artificial; identical to SEQ ID NO: 18
  primer

SEQ ID NO: 29 - DNA, 25 bp, artificial
  primer
agtatgaagggtaggaagaattcgg

SEQ ID NO: 30 - DNA, 28 bp, artificial
  primer
ctaacgtataacgaaaatcgtaacaacc

SEQ ID NO: 31 - DNA, 22 bp, artificial
  primer
cgcgacgtcaaacgccactacg

SEQ ID NO: 32 - DNA, 30 bp, artificial
  primer
cgttatatatcgttcgtagtattcgtgttt

SEQ ID NO: 33 - DNA, 18 bp, artificial
  primer
ctatcgccgcctcatcgt

SEQ ID NO: 34 - DNA, 25 bp, artificial
  primer
ttgggaatttagttgtcgtcgtttc

SEQ ID NO: 35 - DNA, 25 bp, artificial
  primer
aaacaacgactaccgatactacgcg

SEQ ID NO: 36 - DNA, 18 bp, artificial
  primer
acccgaccccgaaccgcg

SEQ ID NO: 37 - DNA, 22 bp, artificial
  primer
tggaattttcggttgattggtt

SEQ ID NO: 38 - DNA, 19 bp, artificial
  primer
aacaacgtccgcacctcct

SEQ ID NO: 39 - DNA, 30 bp, artificial
  primer
ccgacataacccgattaaacccgtacttcg

SEQ ID NO: 40 - DNA, 15 bp, artificial
  primer
cgataccccgcgcga

SEQ ID NO: 41 - DNA, 19 bp, artificial
  primer
gtggtttcgttcggttcgc

SEQ ID NO: 42 - DNA, 23 bp, artificial
  primer
cgacggcgtagggttaagggtcg

SEQ ID NO: 43 - DNA, 20 bp, artificial
  primer
ggattcgcggtgatttacga

SEQ ID NO: 44 - DNA, 26 bp, artificial
  primer
ctacgaaactaaaaaactccgaaacc

SEQ ID NO: 45 - DNA, 26 bp, artificial
  primer
cgttttgaggttcgggtttcgtcgtt

SEQ ID NO: 46 - DNA, 21 bp, artificial
  primer
gggttttggcgagtagtggtc

SEQ ID NO: 47 - DNA, 20 bp, artificial
  primer
gaaaacgaccgacgcgaacg

SEQ ID NO: 48 - DNA, 30 bp, artificial
  primer
ttagaagagagggaaatagggtcgaagcgg

SEQ ID NO: 49 - DNA, 48 bp, artificial
  primer
ttcgcgtgtatttttaggtcggtcgttgtaggatggggtcgcggtcgc

SEQ ID NO: 50 - DNA, 48 bp, artificial
  primer
gttgtaggatggggtcgcggtcgcctactaaccaaactaaaatccaca

SEQ ID NO: 51 - DNA, 26 bp, artificial
  primer
aattttaggttagagggttatcgcgt

SEQ ID NO: 52 - DNA, 19 bp, artificial
  primer
cgcccacccgacctcgcat

SEQ ID NO: 53 - DNA, 22 bp, artificial
  primer
tccccaaaacgaaactaacgac

SEQ ID NO: 54 - DNA, 24 bp, artificial
  primer
cgataaaaccgaacgacccgacga

SEQ ID NO: 55 - DNA, 19 bp, artificial
  primer
ggcgttcgttttgggattg

SEQ ID NO: 56 - DNA, 19 bp, artificial
  primer
gccgacacgcgaactctaa

SEQ ID NO: 57 - DNA, 22 bp, artificial
  primer
cccgtcgaaaacccgccgatta

SEQ ID NO: 58 - DNA, 19 bp, artificial
  primer
gaaccaaaacgctccccat

SEQ ID NO: 59 - DNA, 27 bp, artificial
  primer
ttatatgtcggttacgtgcgtttatat

SEQ ID NO: 60 - DNA, 21 bp, artificial
  primer
gtaatgttttttgatagcggc

SEQ ID NO: 61 - DNA, 15 bp, artificial
  primer
aatcccccttcgccg

SEQ ID NO: 62 - DNA, 24 bp, artificial
  primer
acttcctactacgaccgacgaacc

SEQ ID NO: 63 - DNA, 21 bp, artificial
  primer
tagggaagggggtacgggttt

SEQ ID NO: 64 - DNA, 20 bp, artificial
  primer
cgctcacgaccgtaccctaa

SEQ ID NO: 65 - DNA, 27 bp, artificial
  primer
cgaaaaccctcactcgcgacgaaccgc

SEQ ID NO: 66 - DNA, 20 bp, artificial
  primer
ggtgatttttcggattcggc

SEQ ID NO: 67 - DNA, 20 bp, artificial
  primer
cactcccccgtaaaccgcga

SEQ ID NO: 68 - DNA, 19 bp, artificial
  primer
acgccgcgctcacctccct

SEQ ID NO: 69 - DNA, 18 bp, artificial
  primer
cgacgcaccaacctaccg

SEQ ID NO: 70 - DNA, 18 bp, artificial
  primer
cgacgcaccaacctaccg

SEQ ID NO: 71 - DNA, 25 bp, artificial
  primer
gttttgagttggttttacgttcgtt

SEQ ID NO: 72 - DNA, 28 bp, artificial
  primer
tgattagagatgtaaggggtgaaggagc

SEQ ID NO: 73 - DNA, 23 bp, artificial
  primer
ttacgcgtacgaatttagttaac

SEQ ID NO: 74 - DNA, 25 bp, artificial
  primer
ttttacgcgtacgaatttagttaac

SEQ ID NO: 75 - DNA, 31 bp, artificial
  primer
tcggaacgtattcggaaggttttttgtaagt

SEQ ID NO: 76 - DNA, 18 bp, artificial
  primer
cgagaacgcgagcgattc

SEQ ID NO: 77 - DNA, 24 bp, artificial
  primer
caaacttactcgaccaatccaacc

SEQ ID NO: 78 - DNA, 41 bp, artificial
  primer
tcggtatttagtaggatttattcgatgatagttgttatcgt

SEQ ID NO: 79 - DNA, 27 bp, artificial
  primer
ggtatgtgaacgttattatagtatagc

SEQ ID NO: 80 - DNA, 16 bp, artificial
  primer
ggttagggagggttgc

SEQ ID NO: 81 - DNA, 19 bp, artificial
  primer
aactcgctcgcccgccgaa

SEQ ID NO: 82 - DNA, 22 bp, artificial
  primer
ctctccaaaattaccgtacgcg

SEQ ID NO: 83 - DNA, 21 bp, artificial
  primer
gcgtcggaggttaaggttgtt

SEQ ID NO: 84 - DNA, 21 bp, artificial
  primer
cacgaacgacgccttcccgaa

SEQ ID NO: 85 - DNA, 20 bp, artificial
  primer
caaaccccgctactcgtcat

SEQ ID NO: 86 - DNA, 18 bp, artificial
  primer
gcgcggagcgtagttagg

SEQ ID NO: 87 - DNA, 30 bp, artificial; identical to SEQ ID NO: 21
  primer

SEQ ID NO: 88 - DNA, 25 bp, artificial
  primer
tggtgatggaggaggtttagtaagt

SEQ ID NO: 89 - DNA, 27 bp, artificial
  primer
aaccaataaaacctactcctcccttaa

SEQ ID NO: 90 - DNA, 34 bp, artificial
  primer
acaaccactccctaccacttttaccgcgaactta

SEQ ID NO: 91 - DNA, 17 bp, artificial
  primer
gggggaatcggaagggc

SEQ ID NO: 92 - DNA, 21 bp, artificial
  primer
acccactccttcttcaaaccg

SEQ ID NO: 93 - DNA, 20 bp, artificial
  primer
aggaaggagagagtgcgtcg

SEQ ID NO: 94 - DNA, 29 bp, artificial
  primer
ttaacgacactcttcccttctttcccacg

SEQ ID NO: 95 - DNA, 21 bp, artificial
  primer
cgaataatccaccgttaaccg